(19) United States
(12) Patent Application Publication
(10) Pub. No.: US 2011/0028944 A1
(43) Pub. Date: Feb. 3, 2011

(54) FEMALE URINATION PANTS

(76) Inventors: Chin Hung CHIU, Taichung County (TW); Ju Hui Chiu, Taichung County (TW); Chin Lung Chiu, Taichung County (TW)

Correspondence Address: Chien-Hui Su, P. O. Box 70-121 Taichung, Taichung City, 40899, TW

(21) Appl. No.: 12/904,173

(22) Filed: Oct. 14, 2010

Related U.S. Application Data
(63) Continuation-in-part of application No. 11/943,589, filed on Nov. 21, 2007.

(51) Int. Cl. A61M 27/00

(52) U.S. Cl. 604/544

(57) ABSTRACT

A female urination pants includes a pants body which has an upper opening defined in a top thereof. The pants body has two leg openings defined therein. A crotch portion is formed between the two leg openings. The crotch portion has a receiving hole defined therein. A tubular urine guiding member engages with the receiving hole. The tubular urine guiding member has a hollow receiving unit formed on a first end thereof and a flexible urine draining tube formed on a second end thereof. The hollow receiving unit is funnel-shaped and has a slanted upper rim peripherally formed thereon. A pair of flanges annularly formed on an outer periphery of the tubular urine guiding member for engaging with the receiving hole. At least one securing strap seamed on the pants body for retaining the urine draining tube to the pants body.

### CROSS-REFERENCE TO RELATED APPLICATION

This application is a Continuation-in-part Application of Ser. No. 11/943,589, filed 21 Nov. 2007, and entitled “URINATION PANTS”, now pending.

### BACKGROUND OF THE INVENTION

1. Field of the Invention

The present invention relates to urination pants, and more particularly, to a female urination pants which allows a female user to urinate in a standing pose.

2. Description of Related Art

There are many techniques and devices being employed to enable a female to urinate in a nonsitting position. Some of these methods require a woman to wear an absorbent pad, similar to feminine napkins. Other apparatuses involve the use of an intricate device to control the stream of urine for collection or for disposal in a toilet. Urinary aid devices that work in conjunction with panty-type and belt-type garments are known in the prior art which usually comprises a collection device for female urine and a garment for supporting such device.

However, a drawback of the conventional device is that a shape of the urine collection device does not precisely conform to the contours of the genital region of the user, and the rigid structure of the collection device does not entirely prevent leakage. Also, the panty-type garment does not have an exact means of supporting the rigid structure of the collection device.

Furthermore, no prior art has been disclosed which describes the combined features of the urinary-guide apparatus of the present invention. None of these prior devices or methods employs the combination of components of the present invention, namely, an urinating device made of flexible, semirigid material, which can be cleaned and reused, which contours to the female genital region, and which may be secured and stored in specially constructed garments.

### SUMMARY OF THE INVENTION

The main objective of the present invention is to provide an improved female urination pants.

To achieve the objective, the female urination pants in accordance with the present invention comprises a pants body which has an upper opening defined in a top thereof adapted for enclosing a waist of a user. The pants body has two leg openings defined therein adapted for receiving legs of the user. The two leg openings are located below the upper opening. A crotch portion is formed between the two leg openings. The crotch portion has a receiving hole defined therein. A tubular urine guiding member detachably engages with the receiving hole of the crotch portion. The tubular urine guiding member is made of rubber. The tubular urine guiding member has a hollow receiving unit formed on a first end thereof adapted for guiding urine. The hollow receiving unit is funnel-shaped. The hollow receiving unit has a generally slanted upper rim peripherally formed on a top thereof for invasively fitting in a female genital region to prevent leakage of urine. The slanted upper rim is rounded chamfered for providing a comfort effect to the user. The slanted upper rim has a first tip end and a second tip end opposite to the first tip end.

A second end of the tubular urine guiding member which is opposite to the first end has a flexible urine draining tube formed thereon which is adapted for draining urine. The urine draining tube has a tapered shape. The urine draining tube has at least one protrusion formed thereon. A pair of flanges is annularly formed on an outer periphery of the tubular urine guiding member and positioned between the hollow receiving unit and the urine draining tube for clampingly engaging with the receiving hole in the crotch portion such that the receiving hole is fixed between the pair of flanges for the urine guiding member to be detachably secured with the pants body. A distance between the first tip end of the slanted upper rim of the hollow receiving unit and the pair of flanges is longer than a distance between the second tip end of the slanted upper rim of the hollow receiving unit and the pair of flanges. At least one securing strap is seamed on the pants body for retaining the urine draining tube to the pants body.

When the user urinates, the urine draining tube drains the urine away. When the urine draining tube is not in use, the urine draining tube is bended and securely retained on the pants body by the at least one securing strap.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded perspective view of the female urination pants in accordance with the present invention;

FIG. 2 is an assembled perspective view of the female urination pants in accordance with the present invention;

FIG. 3 is a cross-sectional view of the urine guiding member assembled with the pants body of the female urination pants in accordance with the present invention;

FIG. 4 is a cross-sectional view of the female urination pants in accordance with the present invention, wherein the female urination pants is in a position of use; and

FIG. 5 is another assembled perspective view of the female urination pants in accordance with the present invention, wherein the urine guiding tube is bended and retained on the pants body by at least one securing strap.

### DETAILED DESCRIPTION OF THE INVENTION

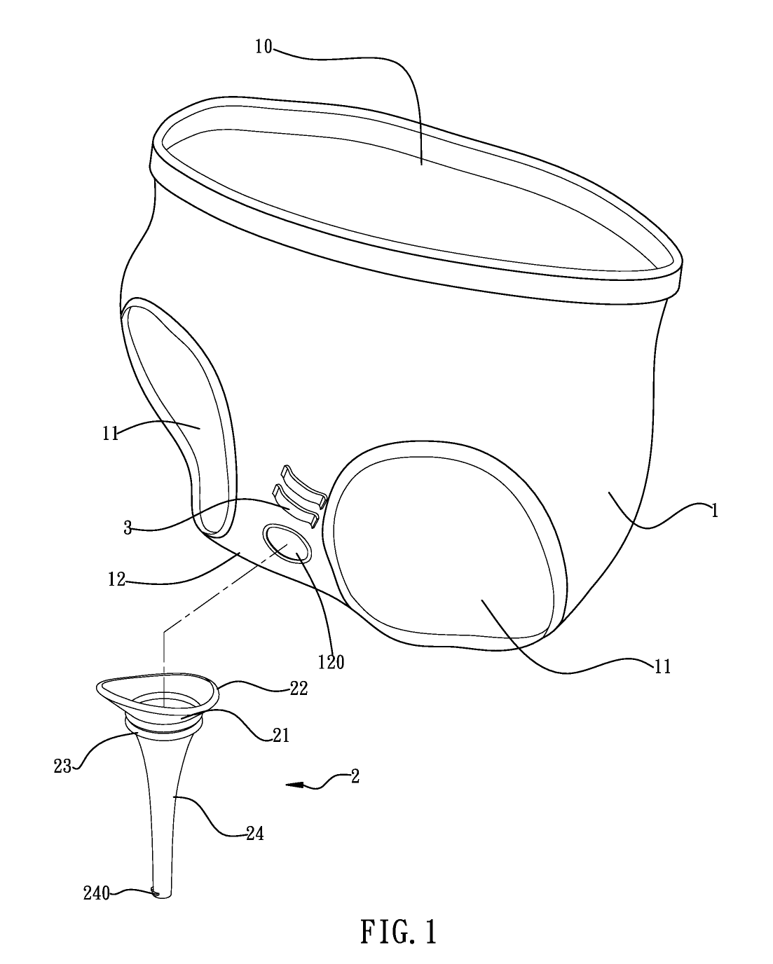

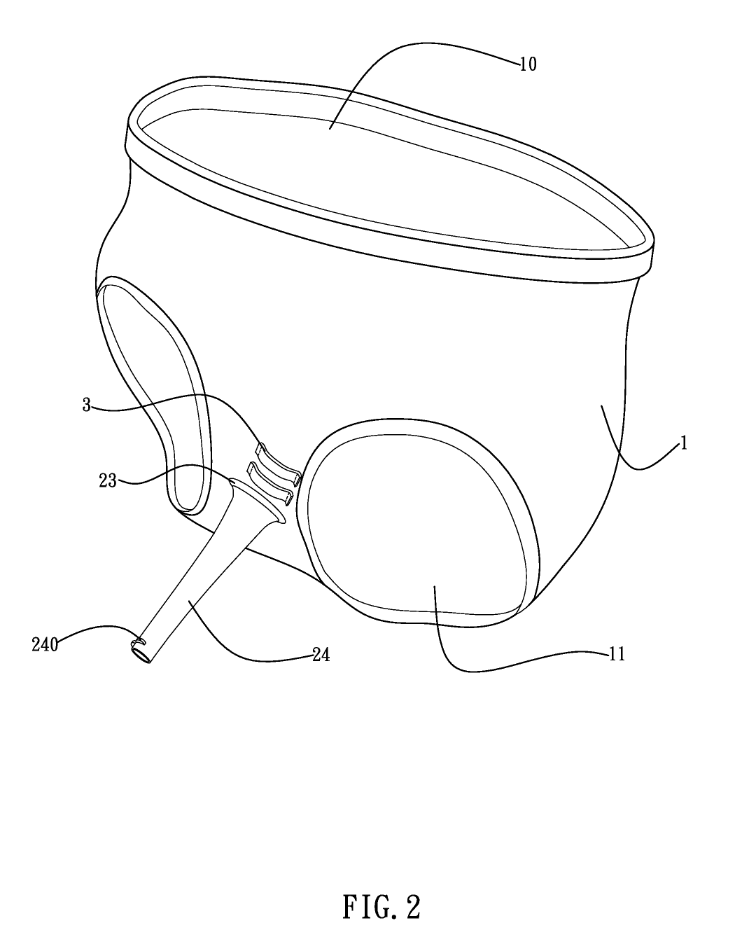

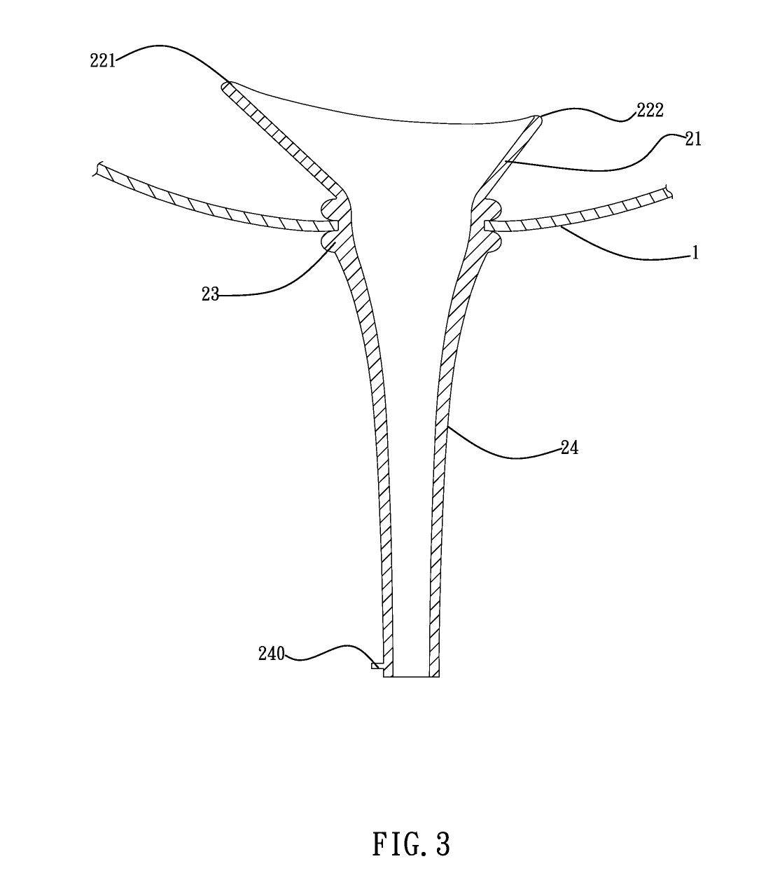

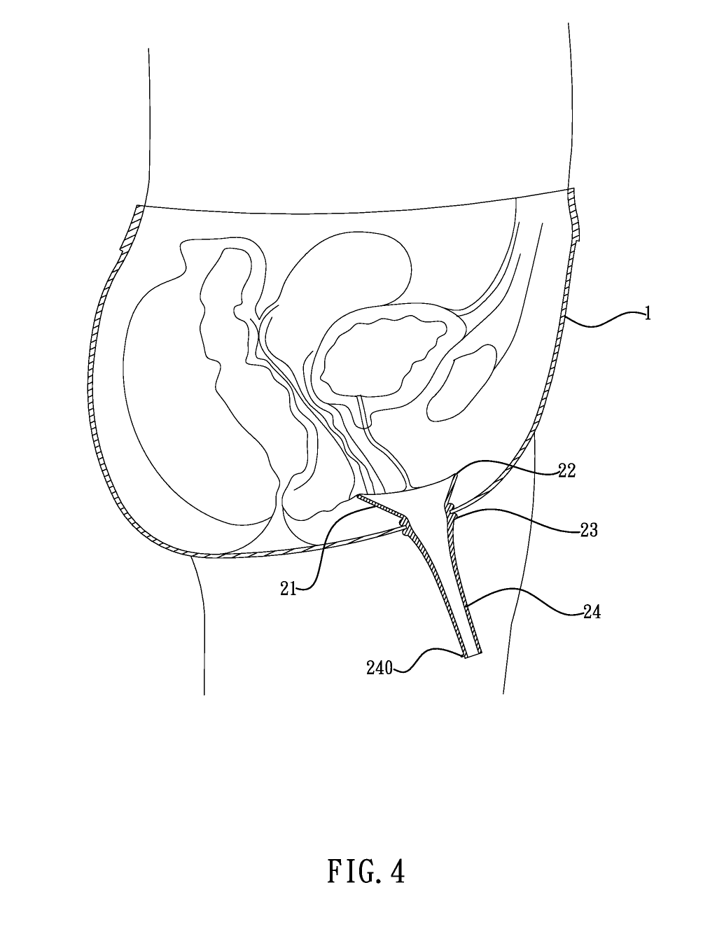

Referring to the drawings and initially to FIGS. 1-4, a female urination pants in accordance with the present invention comprises a pants body 1 which has an upper opening 10 defined in a top thereof adapted for enclosing a waist of a user (not shown). The pants body 1 has two leg openings 11 defined therein adapted for receiving legs of the user. The two leg openings 11 are located below the upper opening 10. A crotch portion 12 is formed between the two leg openings 11. The crotch portion 12 has a receiving hole 120 defined therein. A tubular urine guiding member 2 detachably engages with the receiving hole 120 in the crotch portion 12. The tubular urine guiding member 2 is made of rubber, such that the tubular urine guiding member 2 is semi-rigid yet soft and flexible. The tubular urine guiding member 2 has a hollow receiving unit 21 formed on a first end thereof adapted for guiding urine. The hollow receiving unit 21 is funnel-shaped. The hollow receiving unit 21 has a generally slanted upper rim 22 peripherally formed on a top thereof which is adapted for invasively fitted in a female genital region (not numbered) to prevent leakage of urine. The slanted upper rim 22 has a first tip end 221 and a second tip end 222 opposite to the first tip end 221. As shown in FIG. 3, the slanted upper rim 22 is gradually slanted from the first tip end 221 toward the second tip end 222 for corresponding to a contour of the female genital region. Therefore, as shown in FIG. 4, when in position of use, the first tip end 221 corresponds to a relatively rear portion of the female genital region, wherein the second tip end 222 corresponds to a relatively front portion of the female genital region compares to that of the first tip end 221. Therefore, the slanted upper rim 22 thoroughly embraces the female genital region. The slanted upper rim 22 is rounded chamfered for providing a comfort effect to the user.

A second end of the tubular urine guiding member 2 which is opposite to the first end has a flexible urine draining tube 24 formed thereon which is adapted for draining urine. The urine draining tube 24 has two protrusions 240 formed on a tip thereof. The urine draining tube 24 has a tapered shape. A pair of flanges 23 are annularly formed on an outer periphery of the tubular urine guiding member 23 and positioned between the hollow receiving unit 21 and the urine draining tube 24. A gap (not numbered) is formed between the pair of flanges 23 for clampingly engaging with the receiving hole 120 in the crotch portion 12, such that the receiving hole 120 is fixed between the pair of flanges 23 for the urine guiding member 2 to be detachably secured with the pants body 1. As shown in FIG. 3, a distance between the first tip end 221 of the slanted upper rim 22 of the hollow receiving unit 21 and the pair of flanges 23 is longer than a distance between the second tip end 222 of the slanted upper rim 22 of the hollow receiving unit 21 and the pair of flanges 23. Two securing straps 3 are seamed on the pants body 1 for retaining the urine draining tube 24 to the pants body 1.

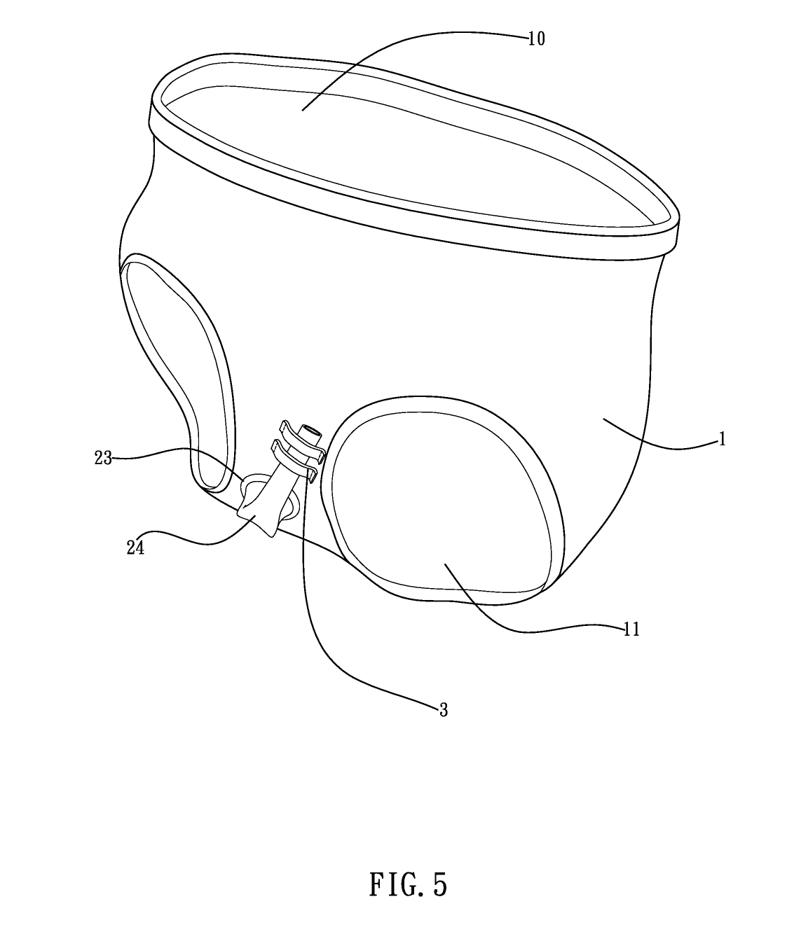

Further referring to FIGS. 4-5, when the user urinates, the urine draining tube 24 drains the urine away; when the urine draining tube 24 is not in use, the urine draining tube 24 is flexibly bended and securely retained on the pants body 1 by the two securing straps 3 (as shown in FIG. 5). Therefore, the urine guiding tube 24 can be easily stored when not is use. The two protrusions 240 of the urine guiding tube 24 abut against the pants body 1 when the urine draining tube 24 is bended for preventing the urine draining tube 24 from contacting with the pants body 1.

Furthermore, as previously mentioned, the urine guiding member 2 is detachably secured on the pants body 1 via the two flanges 23. Therefore, after the urine guiding member 2 has been used, the urine guiding member 2 can be easily removed from the pants body 1 for cleaning purposes such that not only the urine guiding member 2 can be reused for cost-saving purposes, personal hygiene is also effectively maintained. In addition, the urine guiding member 2 is also disposable when it is gradually worn, and a new urine guiding member 2 may be used with the pants body 1, therefore, the present invention is convenient to use and cost-saving.

Although the invention has been explained in relations to its preferred embodiment, it is to be understood that many other possible modifications and variations can be made without departing from the spirit and scope of the invention as hereinafter claimed.

What is claimed is:

1. A female urination pants comprising:
a pants body having an upper opening defined in a top thereof adapted for enclosing a waist of an user; the pants body having two leg openings defined therein adapted for receiving legs of the user, the two leg openings located below the upper opening, a crotch portion formed between the two leg openings, the crotch portion having a receiving hole defined therein;
a tubular urine guiding member detachably engaging with the receiving hole of the crotch portion, the tubular urine guiding member having a hollow receiving unit formed on a first end thereof adapted for guiding urine and a flexible urine draining tube formed on a second end thereof opposite to the first end for draining urine, the hollow receiving unit being funnel-shaped and the urine draining tube having a tapered shape, the hollow receiving unit having a generally slanted upper rim peripherally formed on a top thereof adapted for invasively fitting in a female genital region to prevent leakage of urine;
a pair of flanges annularly formed on an outer periphery of the tubular urine guiding member and positioned between the hollow receiving unit and the urine draining tube for clampingly engaging with the receiving hole in the crotch portion such that the receiving hole is fixed between the pair of flanges for the tubular urine guiding member to be detachably secured with the pants body; and
at least one securing strap seamed on the pants body for retaining the urine draining tube to the pants body;
wherein when the user urinates, the urine draining tube drains the urine away; when the urine draining tube is not in use, the urine draining tube is bended and securely retained on the pants body by the at least one securing strap.

2. The female urination pants as claimed in claim 1, wherein the urine draining tube has at least one protrusion formed thereon for abutting against the pants body to prevent the urine draining tube from contacting with the pants body when the urine draining tube is bended and retained by the at least one strap.

3. The female urination pants as claimed in claim 1, wherein the slanted upper rim has a first tip end and a second tip end opposite to the first tip end; a distance between the first tip end and the pair of flanges is longer than a distance between the second tip end and the pair of flanges.

4. The female urination pants as claimed in claim 1, wherein the slanted upper rim is rounded chamfered for providing a comfort effect to the user.

5. The female urination pants as claimed in claim 1, wherein the tubular urine guiding member is made of rubber.
